(19) United States
(12) Patent Application Publication
(10) Pub. No.: US 2023/0381403 A1
(43) Pub. Date: Nov. 30, 2023

(54) BOX FOR A MEDICAMENT PACKAGING ASSEMBLY

(71) Applicant: Sanofi, Paris (FR)

(72) Inventor: Hardy Kietzmann, Frankfurt am Main (DE)

(21) Appl. No.: 18/031,524

(22) PCT Filed: Oct. 12, 2021
(86) PCT No.: PCT/EP2021/078110

§ 371 Date: Apr. 12, 2023

(30) Foreign Application Priority Data
Oct. 13, 2020 (EP) 20315434.9

(51) Int. Cl. A61M 5/00; B65D 5/42; B65D 5/49; B65D 5/72

(52) U.S. Cl. CPC A61M 5/003; B65D 5/4208; B65D 5/48024; B65D 5/728

(57) ABSTRACT

A box for storing at least one packaging assembly, the packaging assembly configured to receive a plurality of medical devices, the box comprising at least one storage space positioned within a tray, wherein the at least one storage space has a front side and a back side and wherein at least one of the front side or the back side of the storage space comprises at least one opening configured to receive one or more user's fingers and to allow the user to move the at least one packaging assembly stored in the box towards the front side of the box, and the front side of the storage space is configured such that the at least one packaging assembly stored in the box, upon being moved towards the front side, moves at least partially out of the box.

### FIELD

The specification relates to a box for a packaging assembly for a plurality of medical devices and, in particular, although not exclusively, to a cardboard box for the packaging assembly.

### BACKGROUND

Patients suffering chronic disease require regular treatment with medicaments, e.g. on the basis of a predefined schedule. Particular medicaments require refrigerated storage, and are often stored refrigerated in a household refrigerator or fridge. In a home treatment environment, the patient stores the medicament in their fridge or other appropriate location and administers a predefined dose as required. Hence, the medicament is typically provided in a packaging assembly for convenient placement and storage in the household fridge.

Depending on the dosage form of the medicament, the packaging assembly containing the medicament may store a primary packed medicament itself, or may store one or more different kinds of drug delivery devices. For instance, the medicament may be provided in a pre-filled syringe, an auto-injector, or a pen-type injector.

A chronic disease suffered by a patient may cause a loss of mobility, strength and dexterity in the hands and fingers. For example, rheumatoid arthritis is an inflammatory systemic disease of the connective tissue, mainly affecting the joints. Sufferers of rheumatoid arthritis may experience swelling on the fingers and middle joints of the hands, morning stiffness of the fingers and wrists, and pressure pain of the finger rear or toe-rear joints in lateral compression. Patients may be unable to form a closed first or a pinch grip, and may also have reduced strength in the hands and fingers.

To protect the packaging assembly (with or without a medicament) during transport of the packaging assembly from manufacturer to provider and then from provider to the patient's home, the packaging assembly is usually placed in a box, for example a cardboard or plastic box. Conventional boxes used for this purpose, however, may be too difficult for the patient to handle, for example because the patient, due to the loss of mobility, strength and dexterity is unable to grab or manipulate components of the box or the packaging assembly. The patient may thus struggle to open the box, remove all the components of the packaging assembly and accessories from the box, etc.

### SUMMARY

In a first aspect, a box for storing at least one packaging assembly, the packaging assembly configured to receive a plurality of medical devices, the box comprising at least one storage space positioned within a tray, wherein the at least one storage space has a front side and a back side and wherein at least one of the front side or the back side of the storage space comprises at least one opening configured to receive one or more user's fingers and to allow the user to move the at least one packaging assembly stored in the box towards the front side of the box, and the front side of the storage space is configured such that the at least one packaging assembly stored in the box, upon being moved towards the front side, moves at least partially out of the box.

In embodiments of the first aspect, one or more of the following features may be used:
the box further comprises an internal member;
the internal member is fixed within the box;
the internal member is removable from the box;
the internal member is slidable with respect to the box;
the box comprises a ramp adapted to facilitate removal of an item from the box;
the angle of the ramp may be between 30 degrees and 50 degrees, for example less than 45 degrees, and in particular 35 degrees;
the angle of the ramp may be 35 degrees +/−5 degrees and in particular 35 degrees +/−2 degrees;
the box is configured to store at least one medical device and at least one further item;
the at least one medical device and the at least one further item are each stored in an internal member and wherein the internal members are stored on top of each other;
the plurality of medical devices comprises a drug delivery device having at least one ergonomic feature;
the box further comprises a lid, the lid being connected to the tray via a hinge, the lid further comprising a loop opposite the hinge;
the opening is at least 15 mm wide, for example at least 17 mm wide or 17 mm wide;
the opening is 15 mm +/−1 mm wide, or 17 mm +/−2 mm wide, or 17 mm +/−1 mm wide;
the opening is positioned in a second wall of the internal member;
the internal member further comprises at least one opening positioned in a first wall of the internal member opposite the second wall;
the opening in the first wall is configured to allow the item stored in the box to be at least partially moved out of the box;
the storage space comprises at least one lifting feature, the lifting feature being adapted to lift the item stored in the storage space;
the lifting feature is provided as a part of an internal member.

In a second aspect, a method of removing an item stored in a box, the method comprising: opening the box; inserting one or more fingers in the opening; and pushing the packaging assembly towards the front side of the box, thereby moving the packaging assembly at least partially out of the box.

In embodiments of the second aspect, the box may be as described in connection to the first aspect.

In a third aspect, a kit is provided, comprising: a box for storing at least one packaging assembly, the packaging assembly configured to receive a plurality of medical devices, the box comprising at least one storage space positioned within a tray, wherein the at least one storage space has a front side and a back side and wherein at least one of the front side or the back side of the storage space comprises at least one opening configured to receive one or more user's fingers and to allow the user to move the at least one packaging assembly stored in the box towards the front side of the box, and the front side of the storage space is configured such that the at least one packaging assembly stored in the box, upon being moved towards the front side, moves at least partially out of the box, and a packaging assembly configured to receive a plurality of medical devices.

In embodiments of the third aspect, one or more of the following features may be used:
the box further comprises an internal member;
the internal member is fixed within the box;
the internal member is removable from the box;
the internal member is slidable with respect to the box;
the box comprises a ramp adapted to facilitate removal of an item from the box;
the angle of the ramp may be between 30 degrees and 50 degrees, for example less than 45 degrees, and in particular 35 degrees;
the angle of the ramp may be 35 degrees +/−5 degrees and in particular 35 degrees +/−2 degrees;
the box is configured to store at least one medical device and at least one further item;
the at least one medical device and the at least one further item are each stored in an internal member and wherein the internal members are stored on top of each other;
the plurality of medical devices comprises a drug delivery device having at least one ergonomic feature;
the box further comprises a lid, the lid being connected to the tray via a hinge, the lid further comprising a loop opposite the hinge;
the opening is at least 15 mm wide, for example at least 17 mm wide or 17 mm wide;
the opening is 15 mm +/−1 mm wide, or 17 mm +/−2 mm wide, or 17 mm +/−1 mm wide;
the opening is positioned in a second wall of the internal member;
the internal member further comprises at least one opening positioned in a first wall of the internal member opposite the second wall;
the opening in the first wall is configured to allow the item stored in the box to be at least partially moved out of the box;
the storage space comprises at least one lifting feature, the lifting feature being adapted to lift the item stored in the storage space;
the lifting feature is provided as a part of an internal member.

### BRIEF DESCRIPTION OF THE DRAWINGS

Embodiments will now be described, by way of example only, with reference to the accompanying drawings, in which:

FIG. 1A is a box according to the first embodiment wherein the box is closed;

FIG. 1B is a box according to the first embodiment wherein the box is opened and empty;

FIG. 1C is a box according to the first embodiment with first item removed and second item partly removed;

FIG. 2A is a box according to the second embodiment wherein the box is closed;

FIG. 2B is a box according to the second embodiment wherein the box is partly opened and empty;

FIG. 2C is a box according to the second embodiment with first item partly removed and second item partly removed;

FIG. 3A is a box according to the third embodiment wherein the box is closed;

FIG. 3B is a box according to the third embodiment wherein the box is opened and includes two items;

FIG. 3C is a box according to the third embodiment with first item removed and second item partly removed;

FIG. 3D is a tray, used with the box of FIGS. 3A to 3C;

FIG. 4A is an example of item (packaging assembly) stored in the box;

FIG. 4B is an example of a drug delivery device stored in the packaging, which is stored in the box;

FIGS. 5A to 5C are example blanks which may be used to form the boxes of FIGS. 1 to 3;

FIGS. 6A to 6E are example blanks which may be used to form the boxes of FIGS. 1 to 3.

### DETAILED DESCRIPTION

Embodiments provide a box for accommodating and/or protecting a packaging assembly 110, 210, 310, 410 during transportation.

The packaging assembly is configured to contain and store a plurality of drug delivery devices for delivering a medicament. An injection device is an example of a drug delivery device and may be a pen-injector, an auto-injector, a safety system, a pre-filed syringe, or other drug delivery device. The packaging assembly 110, 210, 310, 410 is configured to provide access to the plurality of drug delivery devices 413 for a patient experiencing reduced mobility, strength and/or dexterity. The packaging assembly 110, 210, 310, 410 may further allow for an easy removal or insertion of drug delivery devices 413 by the patient. The packaging assembly 110, 210, 310, 410 provides a predictable, convenient and independent experience for the patient.

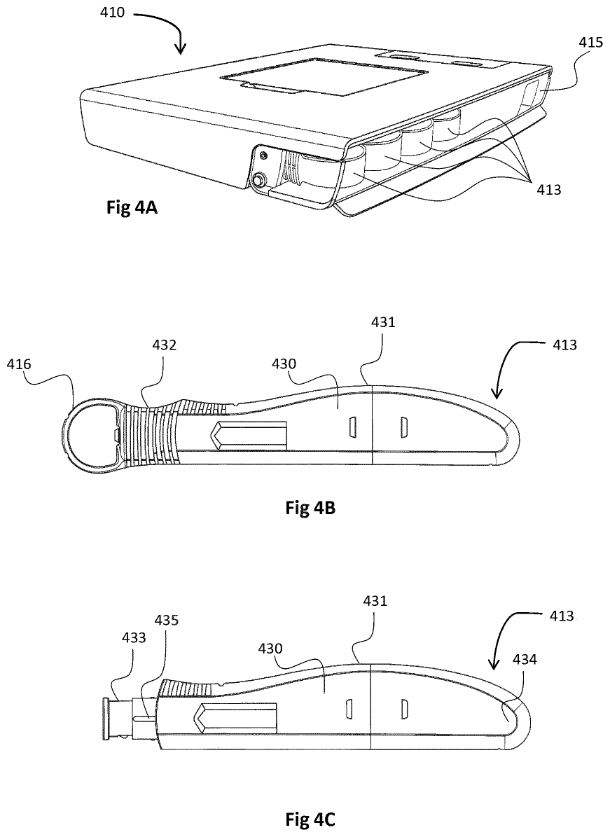

An example of a packaging assembly 410 is shown in FIG. 4A. The packaging assembly 410 may contain a plurality of drug delivery devices 413, stored within the packaging assembly 410. For example, the packaging assembly 410 may store 2, 4 or 6 drug delivery devices 413. The packaging assembly may include a lid 415, for safe and discreet storage of the drug delivery devices. The lid 415 may be configured in such a way that the lid may be opened by the patient without requiring a closed first or pinch grip. The drug delivery device 413 may also be configured for storage within a standard refrigerator. Various attachment subassemblies are contemplated (not shown). Further, drug delivery device 413 may include at least one of a countdown timer, a reset button, a battery and various sensors to conserve battery power.

A drug delivery device 413, the example of which is shown in FIG. 4B, may be configured to be removed from the packaging assembly 410 and handled without requiring a closed first or a pinch grip. The drug delivery device 413 may for example have a loop 416 at its end protruding from the packaging assembly 410. The loop 416 may be configured such that the user may insert their finger through the loop 416 and pull the drug delivery device 413 out of the packaging assembly 410. Such handling may be easier for a user who suffers reduced mobility, strength and dexterity.

The drug delivery device 413, as described above, is configured to inject a medicament into a user's body. The drug delivery device 413 includes a housing 430 which typically contains a reservoir containing the medicament to be injected (e.g., a syringe) and the components required to facilitate one or more steps of the delivery process. As shown, housing 430 is formed in an ergonomic shape. The housing 430 is generally rectangular in shape, with one side face enlarged to form a curved outer surface 431. The curved outer surface 431 improves the fit of the housing in a user's hand, and allows the drug delivery device 413 to be gripped without forming a closed first or a pinch grip. The edges of the housing 430 are generally rounded for user comfort when holding the injection device 413.

The drug delivery device 413 may also include a cap assembly 432 that may be detachably mounted to the housing 430. Typically a user must remove cap 432 from housing 430 before the drug delivery device 413 may be operated. The cap 432 comprises a ring 416 to pull the cap 432 from the housing 430. An internal diameter of the ring 416 is large enough to pass a finger through the ring and pull the cap 432. For example, the internal diameter of the ring 416 may be at least 15 mm, at least 17 mm, at least 20 mm or at least 22 mm. In this way, it is possible to pull the cap 432 from the housing 430 without forming a pinch grip. The enlarged curved outer surface 431 of the housing 430 may prevent the injection device 413 from sliding out of the user's hand when the cap 432 is pulled, even with only a loose grip.

The housing 430 has a distal region 433 and a proximal region 434. The term “distal” refers to a location that is relatively closer to a site of injection, and the term “proximal” refers to a location that is relatively further away from the injection site.

Insertion of needle 435 may occur via several mechanisms. For example, needle 435 may be fixedly located relative to housing 430 and initially be located within an extended needle sleeve. Proximal movement of the sleeve by placing a distal end of the sleeve against a user's body and moving housing 430 in a distal direction will uncover the distal end of needle 435. Such relative movement allows the distal end of needle 435 to extend into the user's body. Such insertion is termed “manual” insertion as needle 435 is manually inserted via the user's manual movement of housing 430 relative to the sleeve.

Another form of insertion is “automated,” whereby needle 435 moves relative to housing 430. Such insertion may be triggered by movement of the sleeve or by another form of activation, such as, for example, a button. A button may be located at a proximal end of housing 430 or, in other embodiments, a button could be located on a side of housing 430.

Other manual or automated features may include drug injection or needle retraction, or both. Injection is the process by which a bung or piston is moved from a proximal location within a syringe (not shown) to a more distal location within the syringe in order to force a medicament from the syringe through needle 435. In some embodiments, a drive spring (not shown) is under compression before device 413 is activated. A proximal end of the drive spring may be fixed within proximal region 434 of housing 430, and a distal end of the drive spring may be configured to apply a compressive force to a proximal surface of the piston. Following activation, at least part of the energy stored in the drive spring may be applied to the proximal surface of the piston. This compressive force may act on the piston to move it in a distal direction. Such distal movement acts to compress the liquid medicament within the syringe, forcing it out of needle 435.

Following injection, needle 435 may be retracted within the sleeve or housing 430. Retraction may occur when the sleeve moves distally as a user removes device 413 from a user's body.

This may occur as needle 435 remains fixedly located relative to housing 430. Once a distal end of the sleeve has moved past a distal end of needle 435, and needle 435 is covered, the sleeve may be locked. Such locking may include locking any proximal movement of the sleeve relative to housing 430.

Another form of needle retraction may occur if needle 435 is moved relative to housing 430. Such movement may occur if the syringe within housing 430 is moved in a proximal direction relative to housing 430. This proximal movement may be achieved by using a retraction spring (not shown), located in distal region 433. A compressed retraction spring, when activated, may supply sufficient force to the syringe to move it in a proximal direction. Following sufficient retraction, any relative movement between needle 435 and housing 430 may be locked with a locking mechanism. In addition, a button or other components of device 413 may be locked as required.

The drug delivery devices 413 stored in the packaging assembly 410 may be configured to inject a medicament into a patient. For example, delivery could be sub-cutaneous, intra-muscular, or intravenous. Such a drug delivery device could be operated by a patient or care-giver, such as a nurse or physician, and may include various types of safety syringe, pen-injector, or auto-injector. The drug delivery device may include a cartridge-based system that requires piercing a sealed ampule before use. Volumes of medicament delivered with these various drug delivery devices may range from about 0.2 ml to about 3 ml.

In combination with a specific medicament, the presently described drug delivery devices may also be customized in order to operate within required specifications. For example, the device may be customized to inject a medicament within a certain time period (e.g., about 3 to about seconds for auto-injectors). Other specifications may include a low or minimal level of discomfort, or to certain conditions related to human factors, shelf-life, expiry, biocompatibility, environmental considerations, etc. Such variations may arise due to various factors, such as, for example, a drug ranging in viscosity from about 3 cP to about 50 cP. Consequently, a drug delivery device will often include a hollow needle ranging from about 25 to about 31 Gauge in size. Common sizes are 27 and 29 Gauge.

The drug delivery devices described herein may also include one or more automated functions. For example, one or more of needle insertion, medicament injection, and needle retraction may be automated. Energy for one or more automation steps may be provided by one or more energy sources. Energy sources may include, for example, mechanical, pneumatic, chemical, or electrical energy. For example, mechanical energy sources may include springs, levers, elastomers, or other mechanical mechanisms to store or release energy. One or more energy sources may be combined into a single device. Devices may further include gears, valves, or other mechanisms to convert energy into movement of one or more components of a device.

The one or more automated functions of an auto-injector may be activated via an activation mechanism. Such an activation mechanism may include one or more of a button, a lever, a needle sleeve, or other activation component. Activation may be a one-step or multi-step process. That is, a user may need to activate one or more activation mechanism in order to cause the automated function. For example, a user may depress a needle sleeve against their body in order to cause injection of a medicament. In other devices, a user may be required to depress a button and retract a needle shield in order to cause injection.

In addition, such activation may activate one or more mechanisms. For example, an activation sequence may activate at least two of needle insertion, medicament injection, and needle retraction. Some devices may also require a specific sequence of steps to cause the one or more automated functions to occur. Other devices may operate with sequence independent steps.

Some drug delivery devices may include one or more functions of a safety syringe, pen-injector, or auto-injector. For example, a drug delivery device could include a mechanical energy source configured to automatically inject a medicament (as typically found in an auto-injector) and a dose setting mechanism (as typically found in a pen-injector).

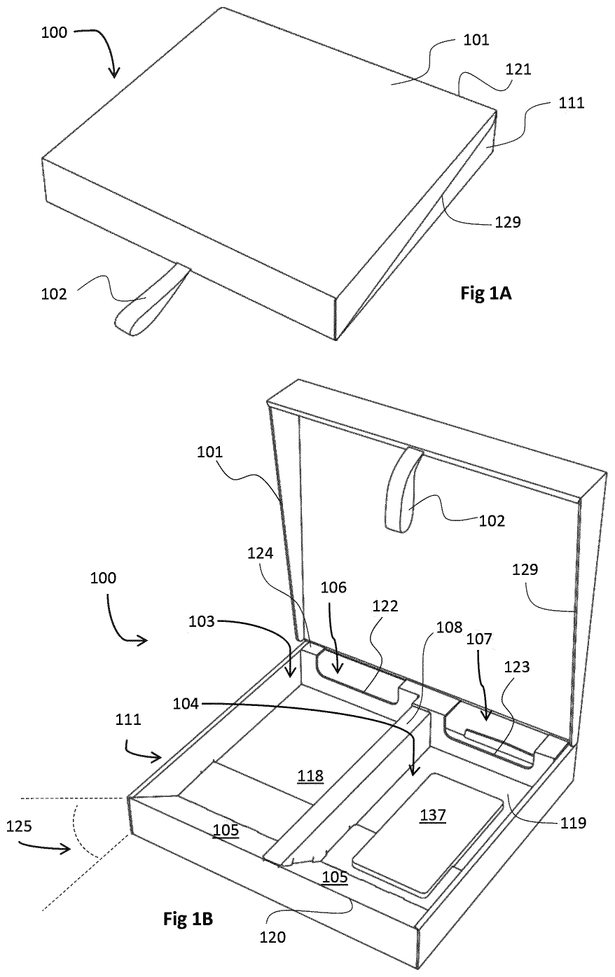

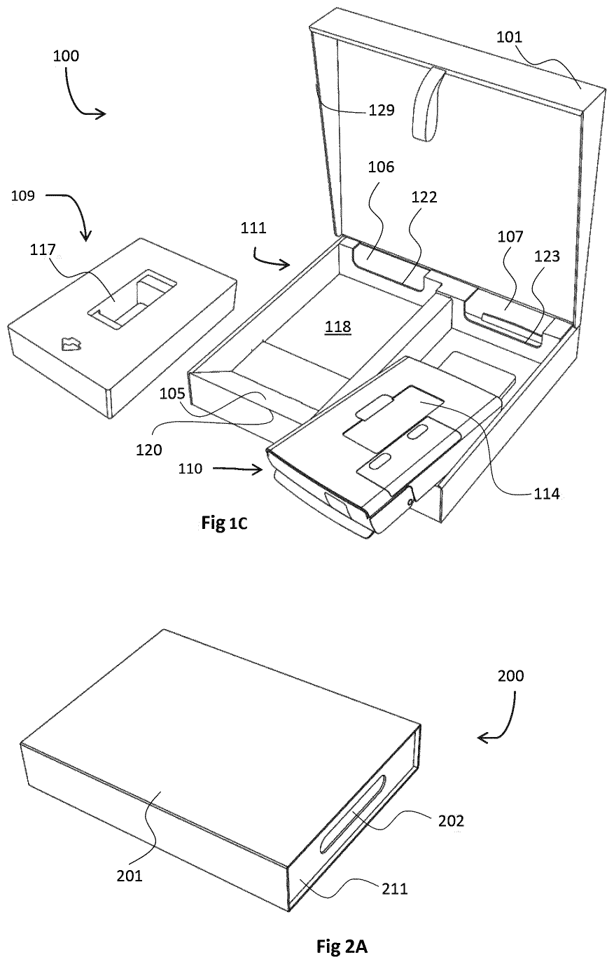

FIGS. 1 to 3 show embodiments of a box 100, 200, 300. The box 100, 200, 300 may be used to protect the packaging assembly and accessories during transportation and handling. The box 100, 200, 300 may be a corrugated box. The box 100, 200, 300 may be made of corrugated fibreboard, corrugated paperboard, cardboard, paperboard (e.g. folding boxboard, chipboard, solid unbleached board, and the like), etc. In some embodiments, the box 100, 200, 300 may be made of a coated laminated cardboard. Such material may show improved properties with respect to strength, waterproofness, etc. The material of the box 100, 200, 300 may be a paper-based material (e.g. as described above) provided with a smooth upper layer. The smooth upper layer may be e.g. a thin plastic coating or a smoothed (polished) paper. More generally, the outer surface of the material may be such that the parts and components of the box (described below) glide when in contact with each other. This may help opening the lid (described below) and/or slide an item out of a tray (described below) and/or slide a storage space provided in the form of a tray (described below in connection with boxes 200, 300) out of the box.

The features of the boxes 100, 200 and 300 of FIGS. 1A to 10, 2A to 2C and 3A to 3D and the advantages of these features generally correspond, as will be apparent from the below. Similar or corresponding features are numbered with corresponding numbers. For the sake of simplicity, some features are described in connection with one embodiment only, but it will be understood that the features of all the embodiments described herein, and in particular the features of the embodiments of FIGS. 1A to 10, 2A to 2C and 3A to 3D may be combined to create an embodiment not described herein.

FIGS. 1A to 10 show the first embodiment of the box 100.

FIG. 1A shows the box 100 in its closed state. FIGS. 1B and 10 show the box 100 with the lid 101 open. FIG. 1B shows the box 100 empty; FIG. 10 shows the box 100 with partly removed items 109, 110.

The box 100 may generally have a tray 111 and a lid 101. The tray 111 and the lid 101 may be connected via a hinge 121, e.g. a living hinge. The side wall of the lid 101 may have an inclined cut 129, making the part of the side wall adjacent the front wall of the box wider than the part of the side wall adjacent the hinge 121. This feature may enable easier opening and closing of the lid 101.

To facilitate opening of the box 100, a loop 102 may be provided on the front side of the lid 101 (i.e. on the side opposite the joint between the lid 101 and the tray 111). The loop 102 is adapted such that a user's finger may be placed inside the loop 102, and the lid 101 may be lifted by means of the loop 102. The loop 102 may be made of a strip of material, e.g. paper, textile or plastics. The strip which forms the loop 102 may be at least 10 cm long.

The tray 111 may comprise at least one internal member. The internal member may provide the tray 111 with an inner structure, for example a first storage space 103, a second storage space 104, a partition 108 or a spacer 124.

The first storage space 103 may store accessories 109, a packaging assembly (not shown), or other items. The tray 111 may further comprise a second storage space 104. The second storage space 104 may store a packaging assembly 110. The packaging assembly 110 may be for example same or similar to the packaging assembly 410 shown in FIG. 4A. The packaging assembly 110 of FIG. 10 may store drug delivery devices same or similar to those shown in FIG. 4B. The packaging assembly stored in the first storage space 103 may be of the same or different type to the first packaging assembly 110.

The first storage space 103 may store accessories 109 of the first packaging assembly 110. Alternatively or in addition, the first storage space 103 may store spare or additional drug delivery devices, a charger, batteries for the first packaging assembly 110, instructions for use of the packaging assembly or a user manual (137), information relating to drug or drugs contained in the drug delivery devices to be stored in the packaging assembly (not shown), and the like.

Instructions for use of the packaging assembly or a user manual (137), information relating to drug or drugs contained in the drug delivery devices to be stored in the packaging assembly (not shown) and other printed material may be stored in the second storage space 104, underneath the item 110. The instructions for use 137 may be provided in a form of a large folded sheet of paper; this may be advantageous for handling of the instructions for use 137 by a user who suffers loss or impairment of mobility, strength and dexterity in the hands and fingers. Additionally, positioning the instructions for use 137 in the bottom of the second storage space 104, underneath the item 110, which lifts the item with respect to the bottom 119 of the second storage space 104, and thus (possibly in combination with all the features described below) may help the user remove the item 110 from the box 100. It should be noted that the item 110 is often larger and/or heavier than the item 109, and thus storing the instructions for use 137 under the item 110 may be of more benefit to certain users than storing it under the item 109.

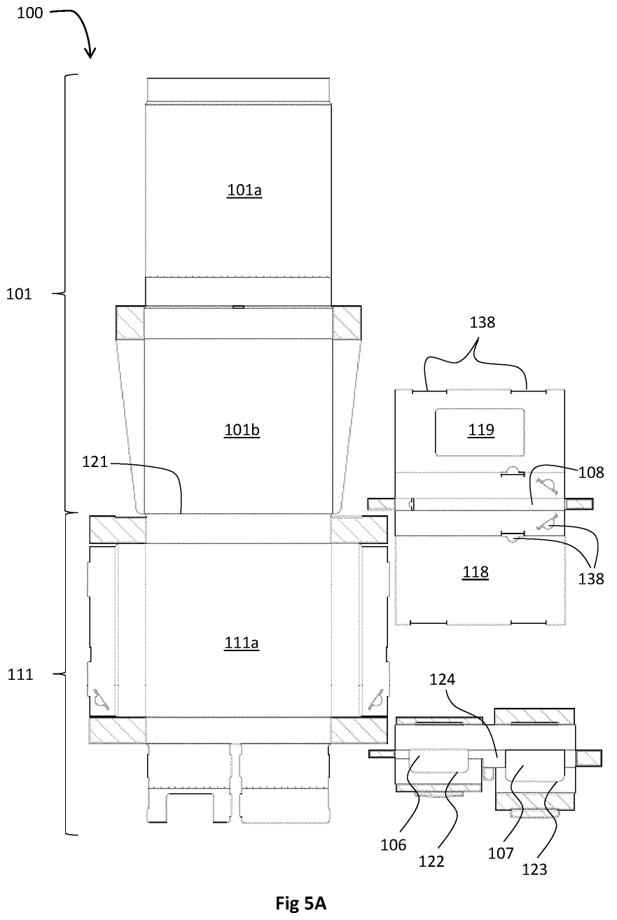

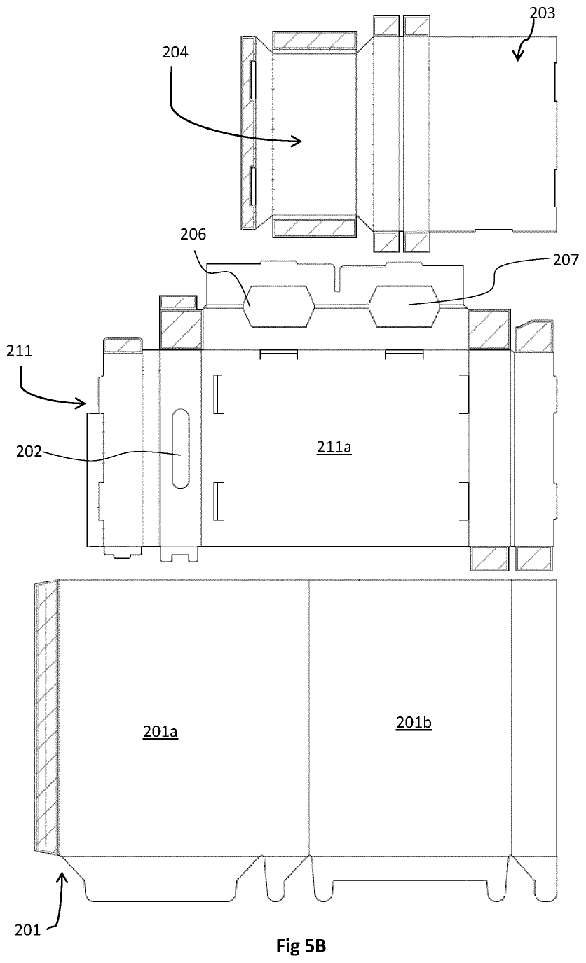

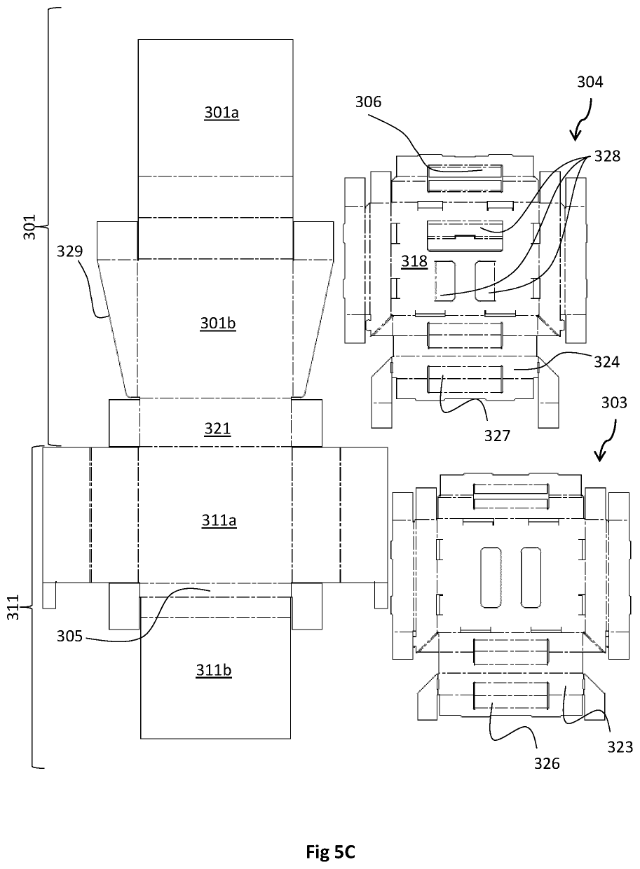

In some embodiments, the second storage space 104 is adapted to store an item 110 which is larger or heavier or both larger and heavier than the item 109 stored in the first storage space 103. For example, the packaging assembly 110 may be heavier than the fixation aid 109. As is apparent from FIG. 5A (described below), the second storage space 104 may be reinforced due to this.

In some embodiments, the accessories 109 may be a fixation aid 109. The fixation aid 109 may carry a fixation plate (not shown) which is adapted to be positioned in a fridge or cupboard and hold the packaging assembly in place. To assist with affixing the fixation plate, the fixation aid 109 may correspond in shape and size to the first packaging assembly 110 and may comprise features which enable to user to position the fixation plate in the fridge or cupboard, such as an enlarged through-hole 117 through which the user may push the fixation plate away from the fixation aid 109. Once the fixation plate is positioned, the packaging assembly 110 may be attached to the fixation plate, for example by means of magnets, positioned in slot 114.

To facilitate removal of the packaging assembly 110 and/or the accessories 109 from the box 100, the lid 101 and the tray 111 may incorporate easy access features. The easy access features are adapted to assist a user who suffers loss or impairment of mobility, strength and dexterity in the hands and fingers. The easy access features may comprise one or more of the loop 102, ramp 105, or openings 106, 107.

The loop 102, described above in connection to FIG. 1A, is an example of an easy access feature. The user may put their finger through the loop 102 and pull upwards to lift and open the lid 101. Fora user with reduced mobility, strength and dexterity in the hands and fingers, using the loop 102 to lift the lid may be easier than opening the lid 101 by grabbing the front side of the lid or the lateral sides of the lid 101.

The ramp 105 may be provided on the front side of the tray 111, i.e. on the side of the tray 111 opposite to the hinge 121 connecting the tray 111 to the lid 101. In the example of FIGS. 1B and 10, the ramp 105 is an inclined surface extending between the bottom face 118, 119 of the first and second storage space 103, 104 towards the front rim 120 of the tray 101.

When an item 109, 110 is to be removed from the first or second storage space 103, 104, respectively, the user does not need to grab the item 109, 110 and lift it up from the respective storage space 103, 104. Instead, the user may push the item 109, 110 out of the respective storage space 103, 104, towards the front rim 120. The ramp 105 facilitates this by allowing the items 109, 110 to slide up the ramp 105 and out of the respective storage space 103, 104. Pushing the item 109, 110 out of the respective storage space 103, 104 via the ramp 105 may be easier for a user with reduced mobility, strength and/or dexterity.

The angle 125 between the ramp 105 and the bottom part 118, 119 of the tray may be less than 50 degrees. In particular, the angle 125 may be less than 45 degrees. The angle 125 may be less than 40 degrees. In some embodiments, the angle 125 is about 35 degrees, for example 35 degrees +/−5 degrees or 35 degrees +/−2 degrees. The angle 125 may be 35 degrees. The angle 125 may be less than 35 degrees, for example 30 degrees. The angle 125 of 35 degrees +/−5 degrees and in particular 35 degrees +/−2 degrees may be advantageous because it allows for the easiest removal of the items 109, 110 from the box 100 while not making the box 100 excessively wide in its front-to-back (width) direction. In addition, the angle 125 of less than 45 degrees allows for easier manufacturing process (e.g. preparing and folding the corrugate blank). Generally, a lower angle leads to a greater simplification of the manufacturing process.

To further facilitate sliding the items 109, 110 from the respective storage spaces 103, 104, openings 106, 107 are provided. The openings 106, 107 are positioned in a spacer 124 provided adjacent the back side of the box 100, i.e. opposite of the ramp 105 and adjacent the hinge 121 connecting the tray 111 and the lid 101. The openings 106, 107 (described in detail below) are provided for the user to put their fingers through the openings 106, 107 and thus enable the user to push the items 109, 110 out of the respective storage space 103, 104.

The spacer 124 extends from the back side of the box 100 towards the inner space of the first and second storage space 103, 104, i.e. towards the front side of the box 100. The spacer 124 may be at least 10 mm wide. In some embodiments, the spacer 124 is at least 15 mm wide. For example, the spacer 124 may be at least 17 mm wide.

In some embodiments, the spacer 124 has a first part and a second part, adjacent to the first and second storage space 103, 104, respectively. The first part (adjacent to the first storage space 103) may have a different width to the second part (adjacent to the second storage space 104). For example, the second part of the spacer 124 may be wider than the first part of the spacer 124. The width of the spacer 124 may be adjusted to accommodate the items 109, 110 stored in the storage spaces 103, 104. For example, if the accessories 109, stored in the first storage space 103, is wider than the packaging assembly 110, stored in the second storage space 104, the part of the spacer 124 adjacent to the first storage space 103 may be narrower than the part of the spacer 124 adjacent to the second storage space 104.

The first opening 106 is provided in the first part of the spacer 124 (adjacent to the first storage space 103). The second opening 107 is provided in the second part of the spacer 124 (adjacent to the second storage space 104). In some embodiments, the openings 106, 107 are provided in the form of U-shaped vertical cut-outs 122, 123 and a horizontal part which has a section with material taken away to allow access into the openings 106, 107. The openings 106, 107 may be at least 15 mm wide, for example 17 mm wide. In some embodiments, the openings 106, 107 may be at least 20 mm wide, at least 25 mm wide, at least 30 mm wide, or at least 35 mm wide. In some embodiments, the width of the openings 106, 107 corresponds to the width of the spacer 124; for example, the width of the section with material taken away is the same as the width of the spacer 124. In general, the width of the openings 106, 107 may be selected such that an average adult male user is able to put their fingers through the openings 106, 107.

The openings 106, 107 enable the user to put their fingers into the openings 106, 107 and, through the U-shaped cut-outs 122, 123, to push the item 109, 110 stored in the first and second storage space 103, 104, respectively, out of the box 100. The height of the U-shaped cut-outs 122, 123 may be at least 10 mm. The height of the U-shaped cut-outs 122, 123 may be at least 15 mm.

In use, the user puts their fingers through one of the openings 106, 107. The space created by the spacer 124 and the openings 106, 107 behind the respective item 109, 110 allows the user to engage the respective item 109, 110 and push the respective item 109, 110 towards the ramp 105. Pushing the item 109, 110 further, the item 109, 110 slides up the ramp 105, protruding at least partly out of the box 100. Like that, the user may easily grab the item 109, 110 and remove it from the box 100, even in case the user suffers reduced mobility, strength and dexterity in the hands and fingers.

The items 109, 110 fit in the box 100 tightly to avoid the items 109, 110 getting damaged by inadvertent movements. Without the ramp 105 and/or the openings 106, 107, the user may struggle to put their fingers through the narrow space between the items 109, 110 and the surrounding walls of the tray 111 and grab the items 109, 110 strongly enough to be able to remove the item 109, 110 from the tray 111, especially if the user's mobility, strength and dexterity in the hands and fingers is reduced. With the features described above, however, this problem is reduced.

To increase the protective function of the box 100 and avoid the items 109, 110 moving, bumping into each other and possibly getting damaged, the tray 111 may comprise a partition 108. The partition 108 divides the inner space of the tray into the first storage space 103 and the second storage space 104 and helps preventing the items stored in first and second storage spaces 103, 104 from inadvertently moving.

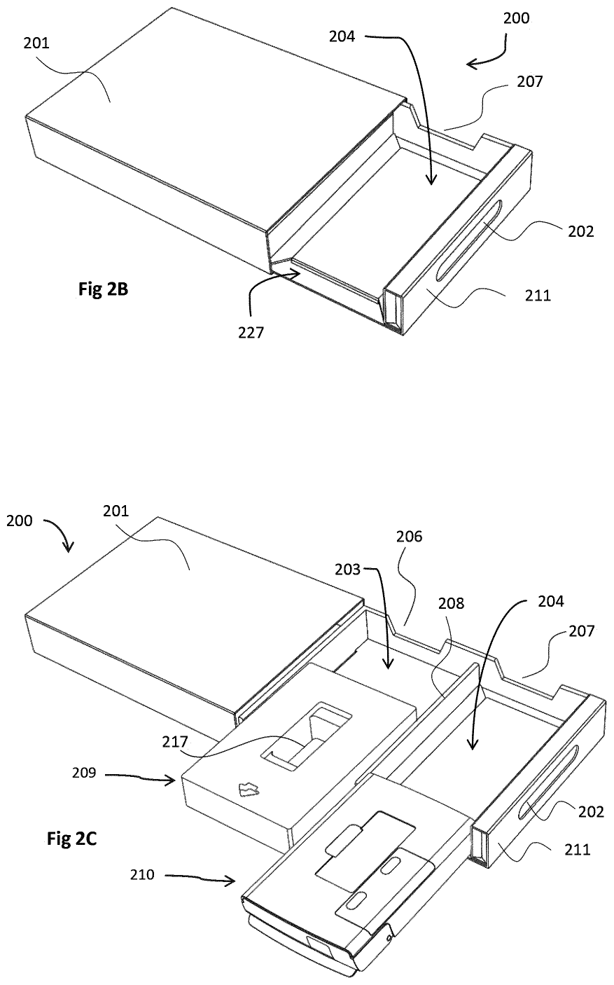

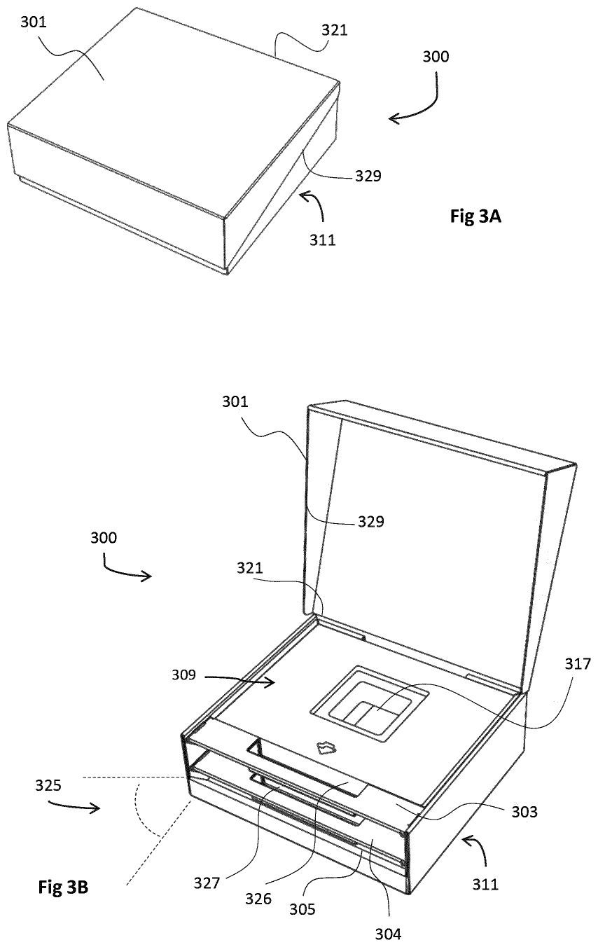

FIGS. 2A to 2C show the second embodiments of the box 200. FIG. 2A shows the box 200 in a closed state. FIGS. 2B and 2C show the box 200 opened, with FIG. 2B showing the box 200 empty and FIG. 2C showing the box 200 with partly removed items 209, 210.

The box 200 comprises a sleeve 201 and a tray 211. The tray 211 may be slidable within the sleeve 201. In the example of FIGS. 2A to 2C, the tray 211 may be slid and pulled out of the sleeve 201. To facilitate sliding the tray 211 out of the sleeve 201, a handle 202 may be provided. The handle 202 may be an opening, for example a through opening, through which the user may insert one or more of their fingers. In use, the user inserts one or more fingers into the opening forming the handle 202 and pulls the tray 211 out of the sleeve.

Alternatively or in addition, a loop (not shown) similar to the loop 102 described in connection with FIGS. 1A to 10 may be provided. The loop has a similar function to loop 102 of FIGS. 1A to 10, and when pulled it may help the user to slide the tray 211 out of the sleeve 201.

Similarly to the box 100 described above in connection to FIGS. 1A to 10, the box 200 may comprise at least one internal member. The tray 211 may be considered to be an internal member. The tray 211 may comprise an internal member. The internal member may provide the box 200 or the tray 211 with an inner structure, for example a first storage space 203, a second storage space 204, a partition 208 or a spacer 224.

The tray 211 may have a first storage space 203 and a second storage space 204. The items 209, 210 stored in the first storage space 203 and the second storage space 204 may be analogous to those items described above in connection with FIGS. 1A to 10. For example, the first storage space 203 may store accessories 209 (which may be of the same type as accessories 109 of FIGS. 1A to 10) and the second storage space 204 may store a packaging assembly 210 (which may be of the same or different type of the packaging assembly 110 of FIGS. 1A to 10).

To facilitate removal of the items 209, 210 stored in the tray 211, the box 200 comprises easy access features such as the handle 202 (described above) and/or a loop (not shown), openings 226, 227 and openings 206, 207.

The tray 201 may have a side wall comprising the handle 202, a side wall opposite the side wall comprising the handle 202, a front wall and a back wall. The front wall and the back wall are disposed between the two side walls and are perpendicular to the side walls.

The front wall comprises two openings 226, 227. The openings 226, 227 provide a passage for the items 209, 210 stored in the first and second storage space 203, 204. The openings 226, 227 may provide a free passage for the items 209, 210. The openings 226, 227 may take a form of two windows, slots, openings or cut-outs in the front wall. The openings 226, 227 are advantageouslywide enough for the items 209, 210 stored in the first and second storage space 203, 204 to pass through the openings 226, 227. Before use, the openings 226, 227 may be covered by a free removable wall (not shown) which may be removed e.g. by putting a pressure on the free removable wall, pulling the free removable wall away from the tray 211, etc.

The openings 206, 207 may be e.g. U-shaped cut-outs provided in the side wall opposite the openings 226, 227. The openings 206, 207 may be wide enough for the user to put their fingers through the openings and push the items 209, 210 through the openings 226, 227 and away from the tray 211. For example, the height of the openings 206, 207 may be about one half of the height of the side wall in which they are formed. For example, the height of the openings 206, 207 may be similar to the cut-outs 122, 123 described in connection to box 100 (e.g. at least 15 mm, as described above).

In use, the user first removes the tray 211 from the sleeve 201, pulling the handle 202 (or the loop (not shown) if provided). Once the tray 211 is removed from the sleeve 201, the user puts their fingers through one of the openings 206, 207 to engage the respective item 209, 210. The user pushes at the item 209, 210 to push the item 209, 210 through the openings 226, 227, provided on the opposite wall to the openings 206, 207. The item 209, 210 slides through the openings 226, 227 and protrudes at least partially from the tray 211. Here, the item 209, 210 may be grabbed by the user.

To increase the protective function of the box 200 and avoid the items 209, 210 bumping into each other, the tray 211 may comprise a partition 208. The partition 208 divides the inner space of the tray into the first storage space 203 and the second storage space 204.

Similarly to the embodiment of FIGS. 1A to 10, the embodiment of FIGS. 2A to 2C helps the user to remove the items 209, 210 from the tray 211 in cases where the user would normally struggle to fit their fingers into narrow spaces between the items 209, 210 and the walls of the tray 211, and/or where the user would struggle to grab the items 209, 210 strongly enough to be able to lift or otherwise remove the items 209, 210 from the tray 211.

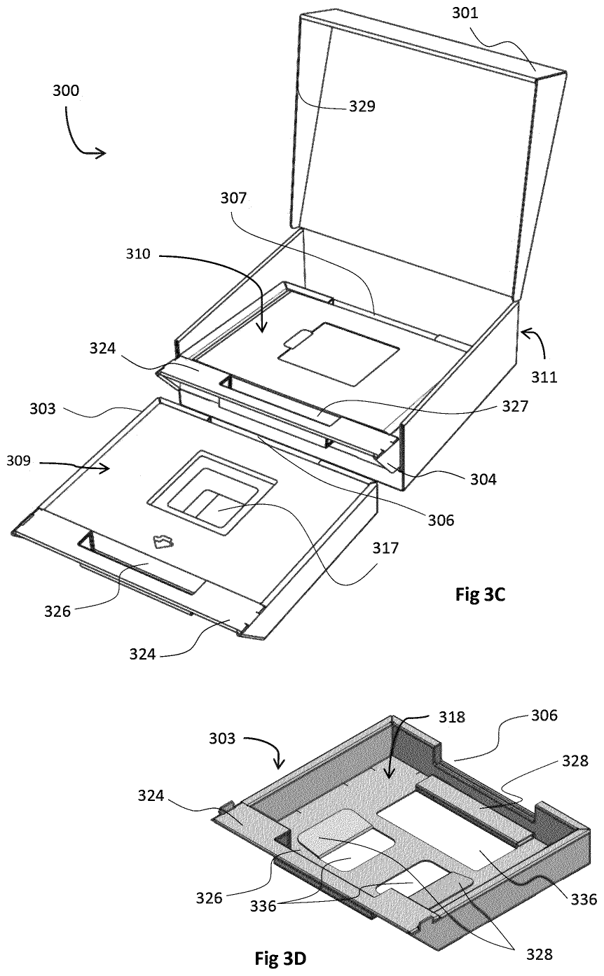

FIGS. 3A to 3D show the third embodiment of the box 300.

The box 300 comprises a lid 301 and a tray 311. The tray 311 and the lid 301 may be connected via a hinge 321, e.g. a living hinge. The side wall of the lid 301 may have an inclined cut 329, making the part of the side wall adjacent the front wall of the box wider than the part of the side wall adjacent the hinge 321. This feature may enable easier opening and closing of the lid 301.

The box 300 may be provided with a loop (not shown) similar to the loop 102 described in connection with FIGS. 1A to 10. Alternatively or in addition, the box 300 may be provided with any other suitable easy access feature such as a handle (not shown).

The box 300 or the tray 311 may comprise at least one internal member. The internal member may provide the tray 311 with an inner structure, for example at least one storage space 303, 304. There may be two internal members, forming a first storage space 303 and a second storage space 304.

The first and second storage spaces 303, 304 may store items 309, 310, respectively, which may correspond to the items 109, 110 stored in the box 100 of FIGS. 1A to 10 and/or items 209, 210 stored in the box 200 of FIGS. 2A to 2C. For example, the first storage space may store accessories 309, and the second storage space 304 may store packaging assembly 310.

The storage spaces 303, 304 may be provided in the form of trays (an example of which is shown in FIG. 3D). Due to the extra layer of cardboard provided by the trays surrounding the packaging assembly 310 and accessories 309, the trays may provide a better protection for the items 309, 310. This may be beneficial for any items stored in the trays, and especially for items which are larger, heavy or both.

The first and second storage spaces 303, 304 may be positioned on top of each other. For example, after opening of the box, the top tray may be the first storage space 303 (called also the first tray 303 hereinafter), storing accessories 309, and the bottom tray (i.e. the tray which rests against the flat bottom of the tray 311) may be the second storage space 304 (also called the second tray 304 hereinafter), storing packaging assembly 310.

Positioning the first and second storage spaces 303, 304 on top of each other, as shown in FIG. 3B, may be advantageous especially in case of storing a larger packaging assembly 310. For example, the packaging assembly 310 may be configured to store six drug delivery devices. Such configuration may make the packaging assembly 310 relatively wide, for example about twice the width of a packaging assembly configured to store two drug delivery devices. If the accessories 309 is fixation aid as described in connection to FIGS. 1a to 10, which may be of comparable width to the packaging assembly 310, positioning the packaging assembly 310 and the accessories 309 next to each other may lead to a box which is more than 60 cm wide. Such width may be inconvenient or impossible for a user to carry. It may therefore advantageous to position the items 309, 310 on top of each other, leading to a box 300 which is higher but less wide.

To remove the items 309, 310 from the box 300, the user may need to first remove the trays 303, 304 which store the items 309, 310. Once the trays 303, 304 are removed, it may suffice to turn each of the trays 303, 304 around so that the bottom of the tray 318 faces upwards and remove the item 309, 310.

To facilitate removal of the first and second trays 303, 304 and thus the items 309, 310 from the box 300, easy access features such as loop (not shown), openings 306, 307 in the back side of the trays 303, 304, openings 326, 327 in the front side of the trays 303, 304 and/or ramp 305 may be provided.

To remove the first storage space (the first tray) 303, an opening 326 may be provided in the extended front portion on the front side of the first tray 303. The opening 326 may be placed centrally with respect to an axis of the box 300 or the first tray 303. The opening 326 may be positioned adjacent to the item 309 placed in the first tray 303. The width of the opening 326 may be the same or similar to width of the openings 106, 107 described in connection with FIGS. 1A to 10. In some embodiments, the opening 326 is wide enough for an average adult male user to be able to put their fingers through the opening 326 and pull the first tray 303 out of the box 300 by means of the opening 326.

In use, the user inserts one or more of their fingers through the opening 326, and pulls the first tray 303 and away from the lid 301. When pulled, the first tray 303 slides along the top of the second tray 304 and the item 310 positioned in the second tray 304 at least until a point where the user may easily grab the first tray 303 with both hands and remove it by further sliding the first tray 303 or lifting the first tray 303 or both to remove it from the box 300.

To remove the second storage space (the second tray) 304, an opening 327 may be provided. The opening 327 may be the same or similar to the opening 326 described above in connection with first tray 303, and it may serve the same purpose, i.e. facilitating removal of the second tray 304 from the box 300.

To further facilitate removal of the second tray 304 from the box 300, the tray 311 of the box 300 may be provided with a ramp 305. The ramp 305 may be similar to the ramp 105 described in connection to FIGS. 1A to 10. The angle 325 (shown in FIG. 3B) may be the same as the angle 125 (shown in FIG. 1B). In some embodiments, the angle 325 may be 45 degrees +/−5 degrees.

The combination of the ramp 305 and the openings 326, 327 in the first and second tray 303, 304, respectively, may be beneficial for easier removal of the first and second tray from the tray 311. In particular, the openings 326, 327 may allow to increase the angle (to e.g. 45 degrees +/−5 degrees), allowing for one of the dimensions of the box 300 (e.g. its depth, i.e. the distance between the front side and the back side of the box 300) to be smaller (shorter).

In use, when the user wishes to remove the second tray 304 from the box 300, the user first removes the first tray 303, as described above. Then, similarly to removing the first tray 303, the user inserts one or more of their fingers through the opening 327 and pulls the second tray 304 and away from the lid 301. The second tray 304 slides along the bottom face of the tray 311 and up the ramp 305; here, the ramp 305 functions similarly to the ramp 105 described in connection to FIGS. 1A to 10. The second tray 304 slides upwards and outwards from the box 300 at least until a point where the user may easily grab the second tray 304 with both hands and remove it by further sliding the second tray 304 or lifting the second tray 304 or both to remove it from the box 300. The initial phase of this is shown in FIG. 3C, which shows the first tray 303 removed from the box 300 and the second tray 304 removed partly from the box 300.

Once the first tray 303 is removed from the box 300, the user may remove the item 309 stored in the first tray 303. To be able to do this easily, the back side of the tray 303 (i.e. the side which, when the box 300 is fully assembled, is positioned adjacent the hinge 321) may comprise an opening 306. The opening 306 may be similar to the opening 206 described above in connection with FIGS. 2A to 2C. Once the tray 303 is removed from the box 303, the user may insert the fingers of one hand through the opening 306 and the fingers of the other hand through the opening 326 to lift the item 309 from the first tray 303.

Similarly, the second tray 304 may be provided with an identical or similar opening 307 which, together with the opening 327, allows the user to grip the item 310.

The height of the openings 306, 307 may be at least one half of the height of the back wall of the first or second tray 303, 304.

Similarly to the boxes 100, 200 described above in connection with FIGS. 1A to 10 and 2A to 2C, the features of the box 300 as described above allow the user to remove the items 309, 310 from the box 300 even in case the user suffers from reduced mobility, strength and/or dexterity. In particular, the features of the box 300 and the first and second trays 303, 304 may help the user to remove the items 309, 310 even in cases when the user is unable to fit their fingers in narrow spaces, form pinch grip, or grab the trays 303, 304 or items 309, 310 stored therein with sufficient force to be able to lift them.

As shown in FIG. 3D, the bottom part 318 of the first tray 303 comprises flaps 328. The flaps may serve as reinforcement features, making the first tray 303 more resilient. The flaps 328 may also act as features which help in facilitating removal of the item 310 from the first tray 303. The flaps 328 may act as springs which push the item 310 out and away from the first tray 303, thus helping the user in removing the item 310. In addition, the apertures 336, left after the flaps 328 have been cut out, may serve as additional features through which the user may put their fingers or a suitable tool, pushing the item 310 out of the first tray 303. It should be noted that the second tray 304 may be provided with the same flaps 328 and apertures 336. These features may help users suffering from reduced mobility, strength and/or dexterity in removing items 309, 310 from the first and second tray 303, 304.

Similarly to the first tray 303, the bottom part of the second tray 304 may also comprise the same flaps 328 as the first tray 303, as described above.

Similarly to what has been described in connection with FIGS. 1A to 10 above, the item 310 stored in the second tray 304 of FIGS. 3A to 3D may be heavier than the item 309 stored in the first tray 303. The second tray 304 may therefore be provided with reinforcement features, as shown in FIG. 3D. The first tray 303 may be provided with less of reinforcement features.

FIGS. 5A to 5C show examples of blanks which may be used to form the boxes 100, 200, 300. In other words, FIGS. 5A to 5C show the example embodiments of FIGS. 1 to 3 in their flat (knocked down, disassembled) state. In FIGS. 5A and 5B, the hatched areas are not visible to the user when the box 100, 200, 300 is in its assembled state.

FIG. 5A shows an example of a blank which may be used to form the box of FIGS. 1A to 10. The reference numerals in FIG. 5A correspond to the reference numerals in FIGS. 1A to 10. The element 101a is an inner layer of the lid 101. The element 101b is an outer layer of the lid 101. The first and second storage spaces 103, 104 and the partition 108 may be provided as a separate inlay (shown on the right hand s ide of FIG. 5A, upper element). Similarly, the spacer 124 together with the openings 106, 107 may be provided as a separate inlay (shown on the right hand s ide of FIG. 5A, lower element). The element 111a is the bottom of the tray 111 onto which the inlay forming the first and second storage spaces 103, 104 and the partition 108 may be positioned. The blank is provided with assembly features 138. These features are an example of paper tabs and corresponding slits that serve in assembly of the blank into the box 100. Other similar features may be used. Alternatively, the box 100 may be glued together.

FIG. 5B shows an example of a blank which may be used to form the box of FIGS. 2A to 2C. The reference numerals in FIG. 5B correspond to the reference numerals in FIGS. 2A to 2C. The element 201a is a bottom layer of the sleeve 201. The element 201b is an outer layer of the sleeve 201. The first and second storage spaces 203, 204 and the partition 208 may be provided as a separate inlay (shown in the uppermost element of FIG. 5B). The element 211a is the bottom of the tray 211 onto which the inlay forming the first and second storage spaces 203, 204 and the partition 208 may be positioned.

FIG. 5C shows an example of a blank which may be used to form the box 300 and the first and second trays 303, 304 of the box 300 of FIGS. 3A to 3D. Similar to FIGS. 5A and 5B, the element 301a is an inner layer of the lid 301 and the element 301b is an outer layer of the lid 301. The element 311a is the outer layer of the bottom of the box 300, and the element 311b is the inner layer of the bottom of the box 300.

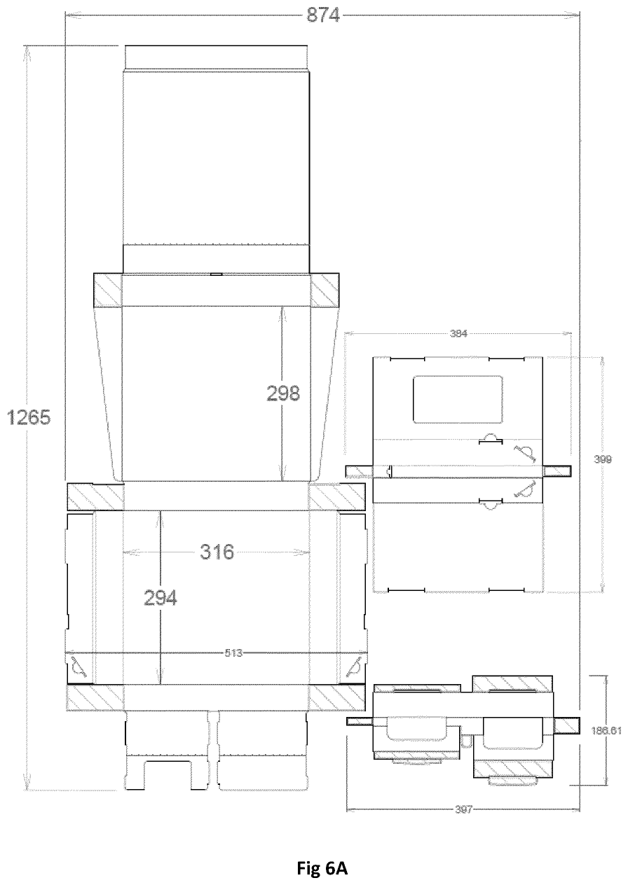

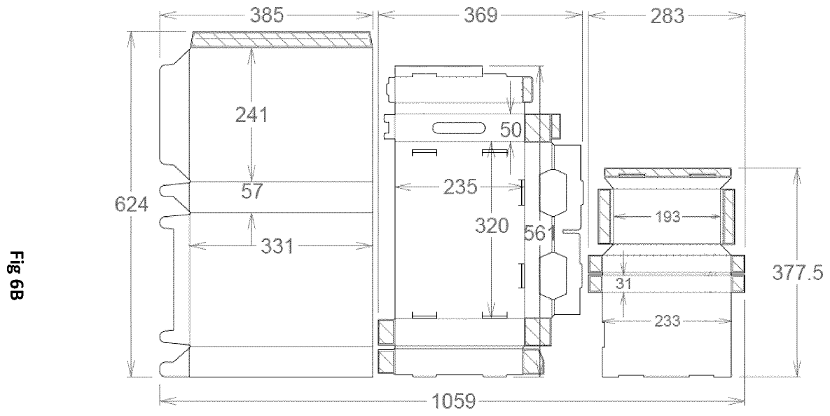

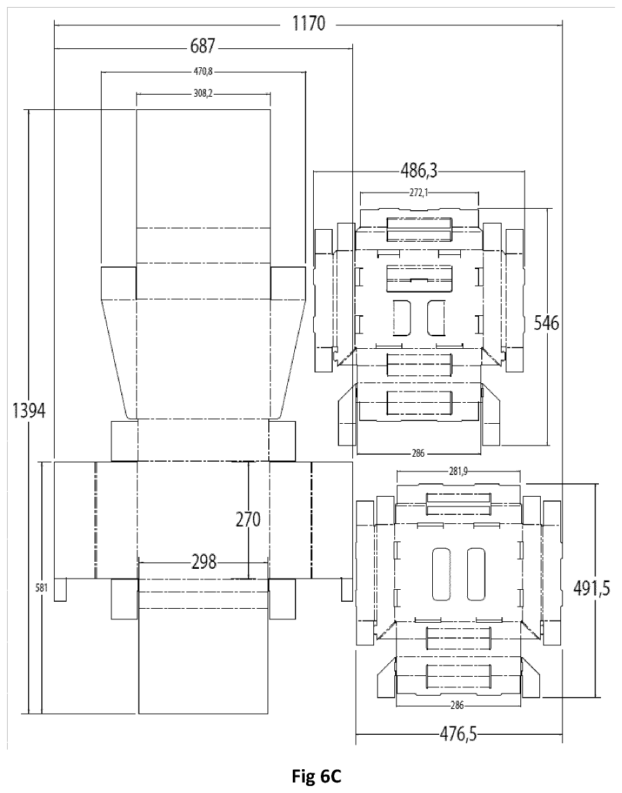

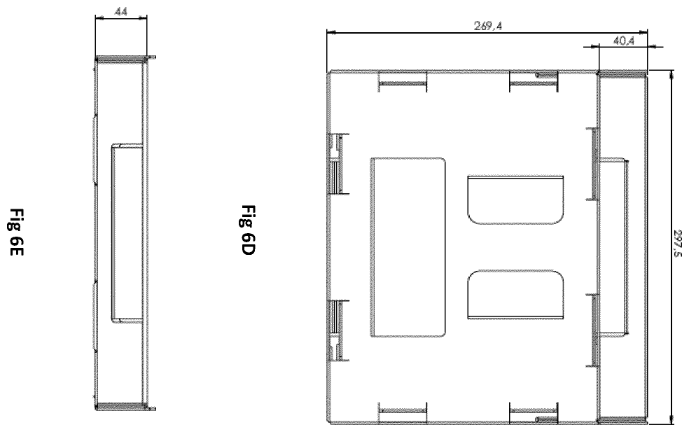

Example embodiments (including dimensions) of the blanks that may serve to assemble boxes 100 and 200 of FIGS. 1A to 10 and 2A to 2C, respectively, are shown on FIGS. 6A and 6B. FIG. 6A corresponds to the more general FIG. 5A, and FIG. 6B corresponds to the more general FIG. 5B.

Similarly, FIGS. 6D and 6E show example embodiments of the blank that may serve to assemble the trays 303, 304 of FIG. 3D. FIG. 6C corresponds to the more general FIG. 5C. FIGS. 6D and 6E are top and back-side view of the tray, respectively.

FIGS. 6A to 6E, and the dimensions given therein, are provided as an example only, and it is to be understood that the subject matter described in this document could be also embodied using a different set of dimensions.

To avoid the components getting stuck or being unduly difficult to open, the dimensions may be kept relatively precise. The required level of precision may depend on the material used (e.g. there may be a difference between corrugated paperboard and smooth plastic). For example, the tolerances (i.e. the allowed deviation from the given dimensions) for all dimensions may be about 0.5 mm. The tolerances for the lid 101, 301 and/or the sleeve 201 may be lower (i.e. these components may be required to be made with more precision), for example 0.4 mm. The tolerances of the inner spaces of the first and second storage spaces or trays 103, 104, 203, 204, 303, 304, i.e. the components holding the items 109, 110, 209, 210, 309, 310, may be even lower, for example 0.3 mm.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

1. A box for storing at least one packaging assembly, the at least one packaging assembly configured to receive a plurality of medical devices, the box comprising at least one storage space positioned within a tray, wherein the at least one storage space has a front side and a back side and wherein:
at least one of the front side or the back side of the storage space comprises at least one opening configured to receive one or more user's fingers and to allow the user to move the at least one packaging assembly stored in the box towards the front side of the box, and
the front side of the storage space is configured such that the at least one packaging assembly stored in the box, upon being moved towards the front side, moves at least partially out of the box.

2. The box of claim 1, further comprising an internal member.

3. The box of claim 2, wherein the internal member is removable from the box.

4. The box of claim 1, wherein the tray comprises a ramp adapted to facilitate removal of an item from the box.

5. The box of claim 4, wherein the angle of the ramp is between 30 degrees and 50 degrees.

6. The box of claim 1, configured to store at least one medical device and at least one further item.

7. The box of claim 2, wherein the internal member is slidable with respect to the box.

8. The box of claim 6, further comprising a plurality of internal members, the plurality of internal members stored on top of each other, wherein the at least one medical device and the at least one further item are each stored in an internal member of the plurality of internal member.

9. The box of claim 1, wherein the opening is at least 15 mm wide.

10. The box of claim 1, wherein the opening is positioned in a second wall of an internal member and wherein the internal member further comprises at least one opening positioned in a first wall of the internal member opposite the second wall, wherein the opening in the first wall is configured to allow the item stored in the box to be at least partially moved out of the box.

11. The box of claim 1, wherein the storage space comprises at least one lifting feature, the lifting feature being adapted to lift the item stored in the storage space.

12. The box of claim 1, wherein the box is made of a single piece of material.

13. The box of claim 12, wherein the single piece of material is one of the group of corrugated fibreboard, corrugated paperboard, cardboard, paperboard.

14. A method of removing an item stored in a box comprising:
opening the box, wherein the box stores at least one packaging assembly configured to receive a plurality of medical devices, wherein the box comprises at least one storage space positioned within a tray, wherein the at least one storage space has a front side and a back side and wherein:
at least one of the front side or the back side of the storage space comprises at least one opening configured to receive one or more user's fingers and to allow the user to move the at least one packaging assembly stored in the box towards the front side of the box, and
the front side of the storage space is configured such that the at least one packaging assembly stored in the box, upon being moved towards the front side, moves at least partially out of the box,
inserting one or more fingers in the at least one opening, and
pushing the at least one packaging assembly towards the front side of the box, thereby moving the at least one packaging assembly at least partially out of the box.

15. A kit comprising:
a box for storing at least one packaging assembly, the box comprising at least one storage space positioned within a tray, wherein the at least one storage space has a front side and a back side and wherein:
at least one of the front side or the back side of the storage space comprises at least one opening configured to receive one or more user's fingers and to allow the user to move the at least one packaging assembly stored in the box towards the front side of the box, and
the front side of the storage space is configured such that the at least one packaging assembly stored in the box, upon being moved towards the front side, moves at least partially out of the box, and
the at least one packaging assembly configured to receive a plurality of medical devices.

16. The kit of claim 15, wherein the box further comprises one or more internal members.

17. The kit of claim 15, wherein the tray comprises a ramp adapted to facilitate removal of an item from the box.

18. The kit of claim 15, wherein the opening is positioned in a second wall of an internal member of the box and wherein the internal member further comprises at least one opening positioned in a first wall of the internal member opposite the second wall, wherein the opening in the first wall is configured to allow the item stored in the box to be at least partially moved out of the box.

19. The kit of claim 15, wherein the storage space comprises at least one lifting feature, the lifting feature being adapted to lift the item stored in the storage space.

20. The kit of claim 15, wherein the box is made of a single piece of material.
